(19) United States
(12) Patent Application Publication
(10) Pub. No.: US 2025/0213314 A1
(43) Pub. Date: Jul. 3, 2025

(54) MICRO-MEDICAL ROBOT-BASED GUIDEWIRE FOR VASCULAR INTERVENTION

(71) Applicants: SEOUL NATIONAL UNIVERSITY HOSPITAL, Seoul (KR); DAEGU GYEONGBUK INSTITUTE OF SCIENCE AND TECHNOLOGY, Daegu (KR); KOSWIRE LTD., Busan (KR)

(72) Inventors: Chang Hwan YOON, Seoul (KR); Hong Soo CHOI, Daegu (KR); Jun Sun HWANG, Daegu (KR); Jae Geun PARK, Busan (KR)

(21) Appl. No.: 18/850,134

(22) PCT Filed: Jan. 27, 2023
(86) PCT No.: PCT/KR2023/001230

§ 371 Date: Sep. 24, 2024

(30) Foreign Application Priority Data
Mar. 25, 2022 (KR) 10-2022-0037686

(51) Int. Cl. A61B 34/30; A61B 34/00; A61L 31/02; A61L 31/08; A61M 25/01; A61M 25/09

(52) U.S. Cl. CPC A61B 34/30; A61L 31/022; A61L 31/088; A61B 2034/301; A61B 2034/731; A61M 25/0127; A61M 25/09041; A61M 2025/09091

(57) ABSTRACT

Disclosed is a micro-medical robot-based guidewire for vascular intervention. The guidewire includes: a core shaft; an outer coil surrounding an outer circumference of a distal portion of the core shaft; a tip end to which a front end of the core shaft and/or a front end of the outer coil is connected; an outer joining unit configured to connect a rear end of the outer coil and a rear end of the core shaft; an inner joining unit, disposed inside the outer coil, configured to connect the outer coil and an outer surface of the core shaft; and at least two magnetic bodies, disposed at a specific section in the inner space of the outer coil, wherein each of the magnetic bodies with each through-hole being formed at each center thereof is configured to allow the front end of the core shaft to go through said each through-hole.

### FIELD OF THE DISCLOSURE

The present disclosure relates to a guidewire; and more particularly to the guidewire that is based on a micro-medical robot for performing vascular intervention.

### BACKGROUND OF THE DISCLOSURE

Vascular treatment using a guidewire is widely used because it is effective in reducing post-procedure recovery time, complications, risk of infection, and postoperative pain. In particular, since vascular interventional procedures can only be performed after the guidewire is inserted into a blood vessel towards a lesion site, a degree of importance of the guidewire in the vascular interventional procedures is very large. During the vascular interventional procedures, the guidewire is controlled by a practitioner using his or her own hands to push and pull the guidewire in order to place it towards the lesion site.

However, according to a prior art disclosed in Korean Patent Publication No. 10-2018-0013838, in case the blood vessel of the lesion site is angled in various ways, it is difficult to insert the guidewire by considering different angles of the blood vessel. Thus, the practitioner should use different guidewires according to the different angles of the blood vessel or should manipulate one guidewire and continuously adjust an angle of the guidewire so that it can be inserted into the different angles of the blood vessel. In the latter case, the practitioner needs to be highly trained, and an operating time becomes long, in expensive labor cost.

Further, since the blood vessel has many uneven curvatures, it is difficult to control a tip end of the guidewire to change a direction thereof, therefore, highly complicated procedures cannot be done, or at the very least, very difficult to be done by using the conventional guidewire.

Therefore, a novel guidewire that can continuously change a direction of the tip end is required to solve the above-mentioned problems.

### SUMMARY OF THE DISCLOSURE

It is an object of the present disclosure to solve all the aforementioned problems.

It is another object of the present disclosure to provide a guidewire including magnetic bodies inside of a front end thereof to allow the front end to change its direction in various ways, resulting in a precise direction control of the guidewire.

It is still another object of the present disclosure to allow a practitioner to control a direction and an angle of the front end of the guidewire by using an external magnetic field system at a branch vessel that is difficult to advance manually, thereby allowing the front end to reach a lesion site faster and with more ease when compared to using a conventional guidewire.

It is still yet another object of the present disclosure to secure a certain gap between each of magnetic bodies disposed inside the guidewire by connecting each of the magnetic bodies through each of inner coils, thereby maintaining a flexibility and a torqueability that are comparable to those of the conventional guidewire even in an absence of a magnetic field.

In order to accomplish objects above, representative structures of the present disclosure are described as follows:

In accordance to one aspect of the present disclosure there is provided a micro-medical robot-based guidewire for performing vascular intervention including: a core shaft; an outer coil configured to surround an outer circumference of a distal portion of the core shaft; a tip end to which at least part of a front end of the core shaft and a front end of the outer coil is connected; an outer joining unit configured to connect a rear end of the outer coil and a rear end of the distal portion of the core shaft; an inner joining unit, disposed in an inner space of the outer coil between the tip end and the outer joining unit, configured to connect the outer coil and an outer surface of the core shaft; and at least two magnetic bodies, disposed at a specific section in the inner space of the outer coil between the tip end and the inner joining unit, wherein each of the magnetic bodies with each through-hole being formed at each center thereof is configured to allow at least part of the front end of the core shaft located at the specific section to go through said each through-hole.

As one example, the magnetic bodies are disposed separately from each other and each of the magnetic bodies are connected via each of inner coils surrounding the core shaft, wherein a first magnetic body among the magnetic bodies is configured to connect directly or indirectly with the tip end and wherein a second magnetic body among the magnetic bodies is configured to connect directly or indirectly with the inner joining unit.

As one example, the magnetic bodies are disposed in one of (i) a first magnetic group axial arrangement in which the first magnetic body is configured to connect directly with the tip end and the second magnetic body is configured to connect directly with the inner joining unit, (ii) a second magnetic group axial arrangement in which the first magnetic body is configured to connect directly with the tip end and the second magnetic body is configured to connect indirectly with the inner joining unit through a second inner coil among the inner coils, wherein a front end of the second inner coil is configured to connect with the second magnetic body and a rear end of the second inner coil is configured to connect with the inner joining unit, (iii) a third magnetic group axial arrangement in which the first magnetic body is configured to connect indirectly with the tip end through a first inner coil among the inner coils, wherein the front end of the first inner coil is configured to connect with the tip end and the rear end of the first inner coil is configured to connect with the first magnetic body, and the second magnetic body is configured to connect directly with the inner joining unit, and (iv) a fourth magnetic group axial arrangement in which the first magnetic body is configured to connect indirectly with the tip end through the first inner coil, wherein the front end of the first inner coil is configured to connect with the tip end and the rear end of the first inner coil is configured to connect with the first magnetic body, and the second magnetic body is configured to connect indirectly with the inner joining unit through the second inner coil, wherein the front end of the second inner coil is configured to connect with the second magnetic body and the rear end of the second inner coil is configured to connect with the inner joining unit.

As one example, the magnetic bodies are disposed in one of (i) a first magnetic group radial arrangement in which each of first air gaps is formed in at least part of sections between each of outer circumferences of the magnetic bodies and the outer coil, and each of second air gaps is formed in at least part of sections between each of inner circumferences of the magnetic bodies and the core shaft, (ii) a second magnetic group radial arrangement in which each of the first air gaps is formed in at least part of sections between each of the outer circumferences of the magnetic bodies and the outer coil, and each of the inner circumferences of the magnetic bodies is configured to connect with the core shaft, (iii) a third magnetic group radial arrangement in which each of the outer circumferences of the magnetic bodies is configured to connect with the outer coil and each of second air gaps is formed in at least part of sections between each of inner circumferences of the magnetic bodies and the core shaft, and (iv) a fourth magnetic group radial arrangement in which each of the outer circumferences of the magnetic bodies is configured to connect with the outer coil and each of the inner circumferences of the magnetic bodies is configured to connect with the core shaft.

As one example, the magnetic bodies are coated in gold.

As one example, (i) a section of the outer coil between the tip end and the inner joining unit is of a first material and (ii) a section of the outer coil between the inner joining unit and the outer joining unit is of a second material.

As one example, the first material is one of Pt alloys and the second material is Stainless Steel.

As one example, a diameter of the outer coil between the tip end and the inner joining unit is greater than a diameter of the outer coil between the inner joining unit and the outer joining unit.

As one example, the core shaft includes at least one tapered part that decreases in a diameter in a direction towards the front end of the core shaft.

As one example, between the inner joining unit and the outer joining unit, a third air gap is formed in at least part of sections between the outer coil and the core shaft.

### BRIEF DESCRIPTION OF THE DRAWINGS

FIG. 1 is a drawing schematically illustrating a cross-sectional view of a guidewire in accordance with one example embodiment of the present disclosure.

FIG. 2 is a drawing schematically illustrating a cross-sectional view of a tip end of the guidewire in accordance with one example embodiment of the present disclosure.

### DETAILED DESCRIPTION OF THE PREFERRED EMBODIMENTS

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the present invention, although different, are not necessarily mutually exclusive. For example, a particular feature, structure, or characteristic described herein in connection with one embodiment may be implemented within other embodiments without departing from the spirit and scope of the present invention.

In addition, it is to be understood that the position or arrangement of individual elements within each disclosed embodiment may be modified without departing from the spirit and scope of the present invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views.

To allow those skilled in the art to carry out the present invention easily, embodiments of the present invention by referring to attached diagrams will be explained in detail as shown below.

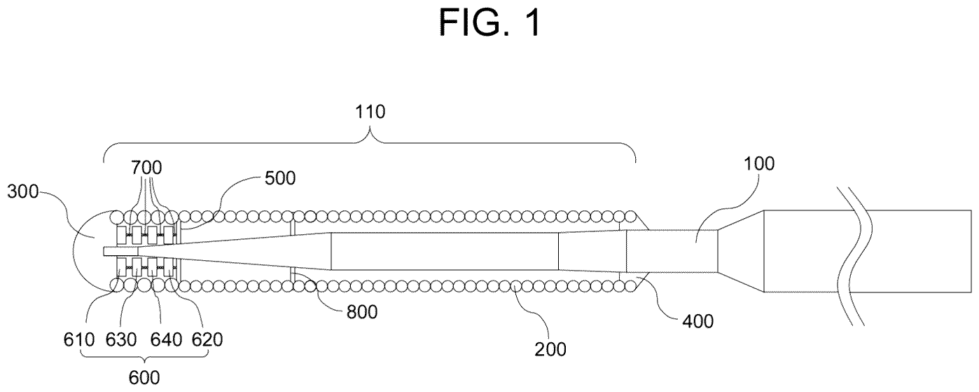

FIG. 1 is a drawing schematically illustrating a cross-sectional view of a guidewire in accordance with one example embodiment of the present disclosure.

Referring to FIG. 1, it can be seen that the guidewire includes a core shaft 100, an outer coil 200, a tip end 300, an outer joining unit 300, an inner joining unit 500, magnetic bodies 600, and inner coils 700. Herein, the magnetic bodies 600 may be a group of at least two magnetic bodies.

First, the core shaft 100 may be a circular rod-like shape made with Stainless Steel, Ni—Ti alloys, nickel-chromium alloys, or cobalt alloys, but it is not limited thereto and other materials with biocompatibility and/or radiopacity may be used.

Herein, the core shaft 100 may be made of a single material, or a combination of different materials. Further, the core shaft 100 may be made to have an overall constant degree of flexibility and rigidity or made to have different degrees of flexibility and rigidity for different parts. For example, a distal portion of the core shaft 100 may be made of a more flexible material than a proximal portion thereof, and the proximal portion thereof may be made of a more rigid material than the distal portion thereof.

Herein, the core shaft 100 may include at least one tapered part whose diameter decreases in a direction towards a front end of the core shaft 100. Herein, the diameter of the tapered part may decrease linearly, stepwise, or non-linearly.

Next, the outer coil 200 may be configured to surround an outer circumference of the distal portion 110 of the core shaft 100.

Herein, the outer coil 200 may surround the outer circumference of the distal portion 110 of the core shaft 100 by (i) spirally winding a single metal wire into a cylindrical shape, (ii) spirally winding strand wire comprised of one metal wire and the other metal wire into the cylindrical shape, or (iii) spirally winding a combination of the single metal wire and the strand wire into the cylindrical shape.

Further, the outer coil 200 may be made with Stainless Steel, tungsten, Ni—Ti alloys, or nickel-chromium alloys, but it is not limited thereto and may be made with other materials that are biocompatible and/or radiopaque.

Next, the tip end 300, to which at least part of a front end of the distal portion 110 of the core shaft 100 and a front end of the outer coil 200 is connected, may be disposed on the front end of the guidewire.

That is, the front end of the distal portion 110 of the core shaft 100 and the front end of the outer coil 200 may be connected to the tip end 300, or the front end of the outer coil 200 may be connected to the tip end 300 but it is not limited thereto. For example, the front end of the distal portion 110 of the core shaft 100 may be spaced apart from the tip end 300 as the case may be.

Herein, a back surface of the tip end 300 to which said at least part of the front end of the distal portion 110 of the core shaft 100 and the front end of the outer coil 300 is connected may be flat, and a front surface of the tip end 300 may be curved like a semi-circle, but it is not limited thereto.

Further, the tip end 300 may be made with gold-tin alloys, silver-tin alloys, silver lead, gold lead, and zinc, but it is not limited thereto and may be made with other biocompatible materials.

Next, the outer joining unit 400 may be configured to connect a rear end of the outer coil 200 and a rear end of the distal portion 110 of the core shaft 100.

Herein, the outer joining unit 400 may be made with gold-tin alloys, silver-tin alloys, silver lead, gold lead, and zinc, but it is not limited thereto and may be made with other biocompatible materials.

Next, the inner joining unit 500 may be disposed in an inner space of the outer coil 200 between the tip end 300 and the outer joining unit 400, and may be configured to connect the outer coil 200 and an outer surface of the core shaft 100.

Herein, the inner joining unit 500 may be made with gold-tin alloys, silver-tin alloys, silver lead, gold lead, and zinc, but it is not limited thereto and may be made with other biocompatible materials.

Next, the magnetic bodies 600 may be disposed at a specific section in the inner space of the outer coil 200 between the tip end 300 and the inner joining unit 500. Herein, each of the magnetic bodies 600 with each through-hole being formed at each center thereof may be configured to allow at least part of the front end of the core shaft 100 located between the tip end 300 and the inner joining unit 500 to go through said each through-hole.

Herein, the magnetic bodies 600 may be magnets, but they are not limited thereto and may be any materials that can be magnetized by the magnetic field.

Further, the magnetic bodies 600 may be spaced apart from each other and each of the magnetic bodies 600 may be connected via each of the inner coils 700 that are surrounding the core shaft 100. Herein, a first magnetic body 610 among the magnetic bodies 600 may be configured to connect directly or indirectly with the tip end 300, and a second magnetic body 620 among the magnetic bodies 600 may be configured to connect directly or indirectly with the inner joining unit 500. Herein, the first magnetic body 610 may be one of the magnetic bodies 600 that is located more closely to the tip end 300 and the second magnetic body 620 may be one of the magnetic bodies 600 that is located more closely to the inner joining unit 500.

That is, the magnetic bodies 600 may be disposed according to one of a first magnetic group axial arrangement to a fourth magnetic group axial arrangement.

Specifically, the magnetic bodies 600 may be disposed according to one of (i) the first magnetic group axial arrangement in which the first magnetic body 610 is configured to connect directly with the tip end 300 and the second magnetic body 620 is configured to connect directly with the inner joining unit 500, (ii) a second magnetic group axial arrangement in which the first magnetic body 610 is configured to connect directly with the tip end 300 and the second magnetic body 620 is configured to connect indirectly with the inner joining unit 500 through a second inner coil among the inner coils 700, wherein a front end of the second inner coil is configured to connect with the second magnetic body 620 and a rear end of the second inner coil is configured to connect with the inner joining unit 500, (iii) a third magnetic group axial arrangement in which the first magnetic body 610 is configured to connect indirectly with the tip end 300 through a first inner coil among the inner coils 700, wherein the front end of the first inner coil is configured to connect with the tip end 300 and the rear end of the first inner coil is configured to connect with the first magnetic body 610, and the second magnetic body 620 is configured to connect directly with the inner joining unit 500, and (iv) the fourth magnetic group axial arrangement in which the first magnetic body 610 is configured to connect indirectly with the tip end 300 through the first inner coil, wherein the front end of the first inner coil is configured to connect with the tip end 300 and the rear end of the first inner coil is configured to connect with the first magnetic body 610, and the second magnetic body 620 is configured to connect indirectly with the inner joining unit 500 through the second inner coil, wherein the front end of the second inner coil is configured to connect with the second magnetic body 620 and the rear end of the second inner coil is configured to connect with the inner joining unit 500.

Herein, each of the inner coils 700 may be comprised of each strand wire made by twisting a single metal wire or a plurality of several metal wires, or comprised of a combination of the strand wire and a metal wire. Further, each of the inner coils 700 may be made with Stainless Steel, tungsten, Ni—Ti alloys, or nickel-chromium alloys, but it is not limited thereto and may be made with other materials that are at least partly biocompatible and/or radiopaque.

Furthermore, the magnetic bodies 600 may be disposed according to one of a first magnetic group radial arrangement to a fourth magnetic group radial arrangement as below by referring to information on whether the magnetic bodies 600 are in contact with the outer coil 200 and the core shaft 100.

Specifically, the magnetic bodies 600 may be disposed according to one of (i) the first magnetic group radial arrangement in which each of first air gaps is formed in at least part of sections between each of outer circumferences of the magnetic bodies 600 and the outer coil 200, and each of second air gaps is formed in at least part of sections between each of inner circumferences of the magnetic bodies 600 and the core shaft 100, (ii) a second magnetic group radial arrangement in which each of the first air gaps is formed in at least part of sections between each of the outer circumferences of the magnetic bodies 600 and the outer coil 200, and each of the inner circumferences of the magnetic bodies 600 is configured to connect with the core shaft 100, (iii) a third magnetic group radial arrangement in which each of the outer circumferences of the magnetic bodies 600 is configured to connect with the outer coil 200 and each of the second air gaps is formed in at least part of sections between each of inner circumferences of the magnetic bodies 600 and the core shaft 100, and (iv) the fourth magnetic group radial arrangement in which each of the outer circumferences of the magnetic bodies 600 is configured to connect with the outer coil 200 and each of the inner circumferences of the magnetic bodies 600 is configured to connect with the core shaft 100.

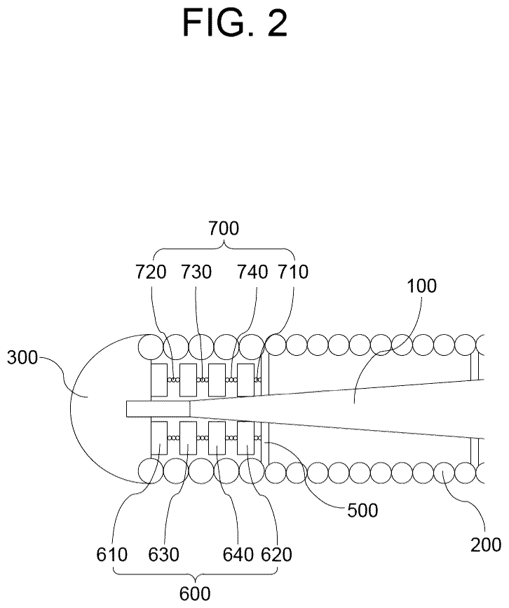

Examples of the magnetic group axial arrangements and the magnetic group radial arrangements of the magnetic bodies 600 are provided by referring to FIG. 2 as below.

FIG. 2 is a drawing schematically illustrating a cross-sectional view of the tip end 300 of the guidewire in accordance with one example embodiment of the present disclosure.

By referring to FIG. 2, it can be seen that magnetic bodies 600 may include the first magnetic body 610, the second magnetic body 620, a third magnetic body 630, and a fourth magnetic body 640. Further, in FIG. 2, the magnetic bodies 600 are arranged according to the second magnetic group axial arrangement in which the first magnetic body 610 is configured to connect directly with the tip end 300 and the second magnetic body 620 is configured to connect indirectly with the inner joining unit 500 through a second inner coil 710 among the inner coils 700, wherein the front end of the second inner coil 710 is configured to connect with the second magnetic body 620 and the rear end of the second inner coil 710 is configured to connect with the inner joining unit 500. Herein, the third magnetic body 630 and the fourth magnetic body 640, which are disposed in between the first magnetic body 610 and the second magnetic body 620, are spaced apart from each other. Further, the third magnetic body 630 is spaced apart from the first magnetic body 610 and the fourth magnetic body 640 is spaced apart from the second magnetic body 620. Furthermore, (i) the first magnetic body 610 and the third magnetic body 630 are connected through an inner coil 720, (ii) the third magnetic body 630 and the fourth magnetic body 640 are connected through an inner coil 730, and (iii) the fourth magnetic body 640 and the second magnetic body 620 are connected through an inner coil 740. Moreover, the magnetic bodies 600 are arranged according to the first magnetic group radial arrangement in which (i) each of the first air gaps is formed between (i-1) each of the outer circumferences of the first magnetic body 610, the second magnetic body 620, the third magnetic body 630, and the fourth magnetic body 640 and (i-2) the outer coil 200, (ii) each of the second air gaps is formed between (ii-1) each of inner circumferences of the first magnetic body 610, the third magnetic body 630, and the fourth magnetic body 640, and (ii-2) the core shaft 100, and (iii) at least part of an inner circumference the second magnetic body 620 is configured to contact directly with the core shaft 100. It is to be appreciated that FIG. 2 illustrates an example of using four magnetic bodies 600, however, the present disclosure is not limited thereto and the number of the magnetic bodies 600 can be determined based on different requirements.

Herein, each of the inner coils 700 may be parallel to the outer coil 200, but it is not limited thereto.

Further, each of diameters of each of the inner coils 700 may be smaller than a diameter of the outer coil 200, but it is not limited thereto.

Meanwhile, the magnetic bodies 600 may be coated in gold. Herein, surfaces of all of the magnetic bodies 600 may be coated in gold, or surfaces of only some of the magnetic bodies 600 may be coated in gold. Through coating the magnetic bodies 600 in gold, a characteristic of radiopacity can be achieved and a bonding strength can be maintained at soldered portions between the magnetic bodies 600 and other parts.

Meanwhile, (i) a section of the outer coil 200 between the tip end 300 and the inner joining unit 500 may be of a first material and (ii) a section of the outer coil 200 between the inner joining unit 500 and the outer joining unit 400 may be of a second material.

Herein, the first material may be one of Pt Alloys and the second material may be Stainless Steel.

Meanwhile, a diameter of the outer coil 200 between the tip end 300 and the inner joining unit 500 may be greater than a diameter of the outer coil 200 between the inner joining unit 500 and the outer joining unit 400.

Meanwhile, between the inner joining unit 500 and the outer joining unit 400, a third air gap may be formed in at least part of sections between the outer coil 200 and the core shaft 100.

For example, since a part of the core shaft 100, which is disposed between the inner joining unit 500 and the outer joining unit 400, may include a part of the tapered part whose diameter decreases towards the front end of the core shaft 100, (i) the outer coil 200 may surround the core shaft 100 except for the tapered part, and thus the third air gap may be formed between the tapered part and the outer coil 200 or (ii) the third air gap may be formed throughout the part of the core shaft 100 disposed between the inner joining unit 500 and the outer joining unit 400 without any contact between the outer coil 200 and the core shaft 100.

Meanwhile, the outer coil 200 may be wound according to one of configurations such as (i) a configuration of each wire unit included in the outer coil 200 being in contact with each other in a longitudinal direction of the core shaft 100, (ii) a configuration of each wire unit included in the outer coil 200 being spaced apart from each other in the longitudinal direction, or (iii) a configuration of some of wire units being in contact with each other in the longitudinal direction and the rest of wire units being spaced apart from each other in the longitudinal direction.

Further, the outer coil 200 may be wound according to one of configurations such as (i) a configuration of each wire unit included in the outer coil 200 being spaced apart evenly with each other in the longitudinal direction or (ii) a configuration of each wire unit included in the outer coil 200 being spaced apart unevenly with each other in the longitudinal direction depending on positions thereof.

Meanwhile, each of the inner coils 700 may be wound according to one of configurations such as (i) a configuration of each wire unit included in each of the inner coils 700 being in contact with each other in the longitudinal direction, (ii) a configuration of each wire unit included in each of the inner coils 700 being spaced apart from each other in the longitudinal direction, or (iii) a configuration of some of wire units of each of the inner coils 700 being in contact with each other in the longitudinal direction and the rest of wire units being spaced apart from each other in the longitudinal direction.

Further, each of the inner coils 700 may be wound according to one of configurations such as (i) a configuration of each wire unit included in each of the inner coils 700 being spaced apart evenly with each other or (ii) a configuration of each wire unit included in each of the inner coils 700 being spaced apart unevenly with each other depending on positions thereof.

Furthermore, each of the inner coils 700 may be wound according to one of styles: (i) the same winding style for all of the inner coils 700, (ii) the same winding style for some of the inner coils 700 and different winding styles for the rest of the inner coils 700, or (iii) different winding styles for all of the inner coils 700.

Meanwhile, the outer coil 200 and each of the inner coils 700 may be wound by using (i) the same winding style for the outer coil 200 and each of the inner coils 700, (ii) the same winding style between the outer coil 200 and some of the inner coils 700 but different winding styles between the outer coil 200 and the rest of the inner coils 700, or (iii) different winding styles for the outer coil 200 and each of the inner coils 700.

Moreover, a winding direction of the outer coil 200 and a winding direction of each of the inner coils 700 may be (i) the same with each other (i) the same between the outer coil 200 and some of the inner coils 700 but different between the outer coil 200 and the rest of the inner coils 700, or (iii) different for the outer coil 200 and each of the inner coils 700.

Meanwhile, by referring to FIG. 1, an inner auxiliary joining unit 800 may be formed between the inner joining unit 500 and the outer joining unit 400 to connect the outer coil 200 and the outer surface of the core shaft 100.

Meanwhile, a coating film may be applied on at least parts of the outer surface of the guidewire.

Herein, the coating film may be made with at least one of a hydrophobic resin and a hydrophilic resin. Herein, the hydrophobic resin may include silicone, polyurethane, polyethylene, polyvinyl chloride, polyester, polypropylene, polyamide, polystyrene, etc., but it is not limited thereto. In addition, the hydrophilic resin may include starch-based substances such as carboxylmethyl starch, cellulose-based substances such as carboxylmethyl cellulose, polysaccharides such as alginic acid, heparin, chitin, chitosan, and hyaluronic acid, and natural water-soluble polymer substances such as gelatin, or synthetic water-soluble polymer substances such as polyvinyl alcohol, polyethylene oxide, polyethylene glycol, polypropylene glycol, polyacrylate, methyl vinyl ether maleic anhydride copolymer, methyl vinyl ether maleic anhydride, methyl vinyl ether maleic anhydride ammonium salt, maleic anhydride ethyl ester copolymer, polyhydroxy ethyl phthalate ester copolymer, polydimethylol propionic acid ester, polyacrylamide, polyacrylamide quaternary compound, polyvinylpyrrolidone, polyethyleneimine, polyethylene sulfonate, and water-soluble nylon, but the present invention is not limited thereto.

As mentioned above, since the magnetic bodies 600 are disposed at the rear part of the front end of the guidewire, the present invention can control a movement of the front end of the guidewire with more precise steering by using an external magnetic field system. In addition, by ensuring a certain gap between each of the magnetic bodies 600 through each of the inner coils 700, the magnetic bodies 600 are prevented from being engaged with each other when the front end of the guidewire is bent, thereby sufficiently maintaining a flexibility and a torqueability.

The present disclosure has an effect of providing the guidewire including the magnetic bodies inside of the front end thereof to allow the front end to change its direction in various ways, resulting in a precise direction control of the guidewire.

The present disclosure has another effect of allowing a practitioner to control a direction and an angle of the front end of the guidewire by using the external magnetic field system at a branch vessel that is difficult to advance manually, thereby allowing the front end to reach a lesion site faster and with more ease when compared to using the conventional guidewire.

The present disclosure has yet another effect of securing the certain gap between each of magnetic bodies disposed inside the guidewire by connecting each of the magnetic bodies through each of the inner coils, thereby maintaining the flexibility and the torqueability that are comparable to those of a conventional guidewire even in an absence of a magnetic field.

As seen above, the present disclosure has been explained by specific matters such as detailed components, limited embodiments, and drawings. They have been provided only to help more general understanding of the present disclosure. It, however, will be understood by those skilled in the art that various changes and modification may be made from the description without departing from the spirit and scope of the disclosure as defined in the following claims.

Accordingly, the thought of the present disclosure must not be confined to the explained embodiments, and the following patent claims as well as everything including variations equal or equivalent to the patent claims pertain to the category of the thought of the present disclosure.

What is claimed is:

1. A micro-medical robot-based guidewire for performing vascular intervention comprising:
a core shaft;
an outer coil configured to surround an outer circumference of a distal portion of the core shaft;
a tip end to which at least part of a front end of the core shaft and a front end of the outer coil is connected;
an outer joining unit configured to connect a rear end of the outer coil and a rear end of the distal portion of the core shaft;
an inner joining unit, disposed in an inner space of the outer coil between the tip end and the outer joining unit, configured to connect the outer coil and an outer surface of the core shaft; and
at least two magnetic bodies, disposed at a specific section in the inner space of the outer coil between the tip end and the inner joining unit, wherein each of the magnetic bodies with each through-hole being formed at each center thereof is configured to allow at least part of the front end of the core shaft located at the specific section to go through said each through-hole.

2. The guidewire of claim 1, wherein the magnetic bodies are disposed separately from each other and each of the magnetic bodies are connected via each of inner coils surrounding the core shaft, wherein a first magnetic body among the magnetic bodies is configured to connect directly or indirectly with the tip end and wherein a second magnetic body among the magnetic bodies is configured to connect directly or indirectly with the inner joining unit.

3. The guidewire of claim 2, wherein the magnetic bodies are disposed in one of (i) a first magnetic group axial arrangement in which the first magnetic body is configured to connect directly with the tip end and the second magnetic body is configured to connect directly with the inner joining unit, (ii) a second magnetic group axial arrangement in which the first magnetic body is configured to connect directly with the tip end and the second magnetic body is configured to connect indirectly with the inner joining unit through a second inner coil among the inner coils, wherein a front end of the second inner coil is configured to connect with the second magnetic body and a rear end of the second inner coil is configured to connect with the inner joining unit, (iii) a third magnetic group axial arrangement in which the first magnetic body is configured to connect indirectly with the tip end through a first inner coil among the inner coils, wherein the front end of the first inner coil is configured to connect with the tip end and the rear end of the first inner coil is configured to connect with the first magnetic body, and the second magnetic body is configured to connect directly with the inner joining unit, and (iv) a fourth magnetic group axial arrangement in which the first magnetic body is configured to connect indirectly with the tip end through the first inner coil, wherein the front end of the first inner coil is configured to connect with the tip end and the rear end of the first inner coil is configured to connect with the first magnetic body, and the second magnetic body is configured to connect indirectly with the inner joining unit through the second inner coil, wherein the front end of the second inner coil is configured to connect with the second magnetic body and the rear end of the second inner coil is configured to connect with the inner joining unit.

4. The guidewire of claim 1, wherein the magnetic bodies are disposed in one of (i) a first magnetic group radial arrangement in which each of first air gaps is formed in at least part of sections between each of outer circumferences of the magnetic bodies and the outer coil, and each of second air gaps is formed in at least part of sections between each of inner circumferences of the magnetic bodies and the core shaft, (ii) a second magnetic group radial arrangement in which each of the first air gaps is formed in at least part of sections between each of the outer circumferences of the magnetic bodies and the outer coil, and each of the inner circumferences of the magnetic bodies is configured to connect with the core shaft, (iii) a third magnetic group radial arrangement in which each of the outer circumferences of the magnetic bodies is configured to connect with the outer coil and each of second air gaps is formed in at least part of sections between each of inner circumferences of the magnetic bodies and the core shaft, and (iv) a fourth magnetic group radial arrangement in which each of the outer circumferences of the magnetic bodies is configured to connect with the outer coil and each of the inner circumferences of the magnetic bodies is configured to connect with the core shaft.

5. The guidewire of claim 1, wherein the magnetic bodies are coated in gold.

6. The guidewire of claim 1, wherein (i) a section of the outer coil between the tip end and the inner joining unit is of a first material and (ii) a section of the outer coil between the inner joining unit and the outer joining unit is of a second material.

7. The guidewire of claim 6, wherein the first material is one of Pt alloys and the second material is Stainless Steel.

8. The guidewire of claim 1, wherein a diameter of the outer coil between the tip end and the inner joining unit is greater than a diameter of the outer coil between the inner joining unit and the outer joining unit.

9. The guidewire of claim 1, wherein the core shaft includes at least one tapered part that decreases in a diameter in a direction towards the front end of the core shaft.

10. The guidewire of claim 1, wherein, between the inner joining unit and the outer joining unit, a third air gap is formed in at least part of sections between the outer coil and the core shaft.
